SEQUENCE LISTING (47 sequences)

SEQ ID NO: 1 - protein, 50 aa, artificial
  HXL008 Picramnia pentandra
KVCTKPSKFFKGLCGTDGACTTACRKEGLHSGYCQLKGFLNSVCVCRKHC

SEQ ID NO: 2 - protein, 50 aa, artificial
  HXL035 Picramnia pentandra
KVCTKPSKFFKGLCGFDRDCTVACKKEGLASGFCQNKGFFNVVCVCRKPC

SEQ ID NO: 3 - protein, 50 aa, artificial
  HXL036 Picramnia pentandra
KVCTKPSKFFKGLCGADRDCTVACKKEGLATGFCQKKGFFNFVCVCRKPC

SEQ ID NO: 4 - protein, 49 aa, artificial
  HXL001 Zea mays
RHCLSQSHRFKGLCMSSNNCANVCQTENFPGGECKAEGATRKCFCKKIC

SEQ ID NO: 5 - protein, 49 aa, artificial
  HXL002 Triticum aestivum
RDCLSQSHKFKGACLSSSNCAAVCRTENFPDGECHTHNFARKCFCKRAC

SEQ ID NO: 6 - protein, 46 aa, artificial
  HXL003 Triticum aestivum
ARDCTSQSHKFVGLCLSDRNCASVCLTEYFTGGKCDHRRCVCTKGC

SEQ ID NO: 7 - protein, 47 aa, artificial
  HXL004 Nicotiana benthamiana
RTCESQSHRFKGLCFSRSNCASVCHTEGFNGGHCRGFRRRCFCTRHC

SEQ ID NO: 8 - protein, 45 aa, artificial
  HXL005 Taraxcum kok-saghyz
KMCQTTSHAFSCVNDSGCSGSCEKQGFASGKCDGVRRRCTCYKKC

SEQ ID NO: 9 - protein, 51 aa, artificial
  HXL009 Zea mays
TVCMRHNNFYHGPCMSNKDCANSCVQHNLGVGGYCRGKIPFNKECMCTFEC

SEQ ID NO: 10 - protein, 48 aa, artificial
  HXL012 Amaranthus retroflexus
RMCKAPSKLFRGMCGIRDSNCDSVCRAEGMAAGDCHGLRRRCICSRPC

SEQ ID NO: 11 - protein, 47 aa, artificial
  HXL013 Glycine max
KDCLTRRHGFQGRCLFDRQCAHVCRSDGFIGGQCRGPLRKCFCSRPC

SEQ ID NO: 12 - protein, 49 aa, artificial
  HXL015 Oryza sativa
RHCLSQSHRFKGMCVSSNNCANVCRTESFPDGECKSHGLERKCFCKKVC

SEQ ID NO: 13 - protein, 49 aa, artificial
  HXL032 Triticum aestivum
RTCLSQSHKFKGTCLSNSNCAGVCRTENFPDGECNSHRLERKCFCKRTC

SEQ ID NO: 14 - protein, 47 aa, artificial
  HXL033 Parthenium argentatum
RTCESQSHKFKGTCLSDTNCGNVCHSEGFPGGKCRGLRRRCFCTKNC

SEQ ID NO: 15 - protein, 47 aa, artificial
  HXL034 Nicotiana benthamiana
RRCESKSQRFKGPCVRVKNCAAVCETEGFSGGDCRGLRRRCFCTRPC

SEQ ID NO: 16 - protein, 47 aa, artificial
  NsD1 Nicotiana suaveolens
KDCKRESNTFPGICITKPPCRKACIREKFTDGHCSKILRRCLCTKPC

SEQ ID NO: 17 - protein, 47 aa, artificial
  NsD2 Nicotiana suaveolens
KDCKRESNTFPGICITKLPCRRACISEKFADGHCSKILRRCLCTKPC

SEQ ID NO: 18 - protein, 47 aa, artificial
  NaD1 Nicotiana alata
RECKTESNTFPGICITKPPCRKACISEKFTDGHCSKILRRCLCTKPC

SEQ ID NO: 19 - protein, 47 aa, artificial
  NoD173 Nicotiana occidentalis spp obliqua
RQCKAESNTFTGICIAKPPCRQACIREKFTDGHCSKVLRRCLCTKRC

SEQ ID NO: 20 - protein, 50 aa, artificial
  DmAMP1 Dahlia merckii
ELCEKASKTWSGNCGNTGHCDNQCKSWEGAAHGACHVRNGKHMCFCYFNC

SEQ ID NO: 21 - protein, 47 aa, artificial
  HXP4
RECKTESHRFKGPCITKPPCRKACISEKFTDGHCSKILRRCLCTKPC

SEQ ID NO: 22 - protein, 47 aa, artificial
  HXP34
RECKTESQHHSFPCITKPPCRKACISEKFTDGHCSKILRRCLCTKPC

SEQ ID NO: 23 - protein, 47 aa, artificial
  HXP35
RECKTESDTYRGVCITKPPCRKACISEKFTDGHCSKILRRCLCTKPC

SEQ ID NO: 24 - protein, 51 aa, artificial
  Consensus
  MISC_FEATURE (1)..(1): X is optionally alanine or no residue
  MISC_FEATURE (2)..(51): X is any amino acid
XKVCTKPSKFFKGLCGXDXXCTXACXKEGLXXGXCQXKGFXNXVCVCRKXC

SEQ ID NO: 25 - protein, 51 aa, artificial
  HXL008 + N-terminal alanine
AKVCTKPSKFFKGLCGTDGACTTACRKEGLHSGYCQLKGFLNSVCVCRKHC

SEQ ID NO: 26 - protein, 51 aa, artificial
  HXL035  + N-terminal alanine
AKVCTKPSKFFKGLCGFDRDCTVACKKEGLASGFCQNKGFFNVVCVCRKPC

SEQ ID NO: 27 - protein, 51 aa, artificial
  HXL036  + N-terminal alanine
AKVCTKPSKFFKGLCGADRDCTVACKKEGLATGFCQKKGFFNFVCVCRKPC

SEQ ID NO: 28 - protein, 50 aa, artificial
  HXL001 + N-terminal alanine
ARHCLSQSHRFKGLCMSSNNCANVCQTENFPGGECKAEGATRKCFCKKIC

SEQ ID NO: 29 - protein, 50 aa, artificial
  HXL002  + N-terminal alanine
ARDCLSQSHKFKGACLSSSNCAAVCRTENFPDGECHTHNFARKCFCKRAC

SEQ ID NO: 30 - protein, 46 aa, artificial; identical to SEQ ID NO: 6
  HXL003  + N-terminal alanine

SEQ ID NO: 31 - protein, 48 aa, artificial
  HXL004  + N-terminal alanine
ARTCESQSHRFKGLCFSRSNCASVCHTEGFNGGHCRGFRRRCFCTRHC

SEQ ID NO: 32 - protein, 45 aa, artificial; identical to SEQ ID NO: 8
  HXL005  + N-terminal alanine

SEQ ID NO: 33 - protein, 52 aa, artificial
  HXL009  + N-terminal alanine
ATVCMRHNNFYHGPCMSNKDCANSCVQHNLGVGGYCRGKIPFNKECMCTFEC

SEQ ID NO: 34 - protein, 49 aa, artificial
  HXL012  + N-terminal alanine
ARMCKAPSKLFRGMCGIRDSNCDSVCRAEGMAAGDCHGLRRRCICSRPC

SEQ ID NO: 35 - protein, 48 aa, artificial
  HXL013  + N-terminal alanine
AKDCLTRRHGFQGRCLFDRQCAHVCRSDGFIGGQCRGPLRKCFCSRPC

SEQ ID NO: 36 - protein, 50 aa, artificial
  HXL015  + N-terminal alanine
ARHCLSQSHRFKGMCVSSNNCANVCRTESFPDGECKSHGLERKCFCKKVC

SEQ ID NO: 37 - protein, 50 aa, artificial
  HXL032  + N-terminal alanine
ARTCLSQSHKFKGTCLSNSNCAGVCRTENFPDGECNSHRLERKCFCKRTC

SEQ ID NO: 38 - protein, 48 aa, artificial
  HXL033  + N-terminal alanine
ARTCESQSHKFKGTCLSDTNCGNVCHSEGFPGGKCRGLRRRCFCTKNC

SEQ ID NO: 39 - protein, 48 aa, artificial
  HXL034  + N-terminal alanine
ARRCESKSQRFKGPCVRVKNCAAVCETEGFSGGDCRGLRRRCFCTRPC

SEQ ID NO: 40 - protein, 48 aa, artificial
  NsD1   + N-terminal alanine
AKDCKRESNTFPGICITKPPCRKACIREKFTDGHCSKILRRCLCTKPC

SEQ ID NO: 41 - protein, 48 aa, artificial
  NsD2  + N-terminal alanine
AKDCKRESNTFPGICITKLPCRRACISEKFADGHCSKILRRCLCTKPC

SEQ ID NO: 42 - protein, 48 aa, artificial
  NaD1  + N-terminal alanine
ARECKTESNTFPGICITKPPCRKACISEKFTDGHCSKILRRCLCTKPC

SEQ ID NO: 43 - protein, 48 aa, artificial
  NoD173  + N-terminal alanine
ARQCKAESNTFTGICIAKPPCRQACIREKFTDGHCSKVLRRCLCTKRC

SEQ ID NO: 44 - protein, 51 aa, artificial
  DmAMP1  + N-terminal alanine
AELCEKASKTWSGNCGNTGHCDNQCKSWEGAAHGACHVRNGKHMCFCYFNC

SEQ ID NO: 45 - protein, 48 aa, artificial
  HXP4  + N-terminal alanine
ARECKTESHRFKGPCITKPPCRKACISEKFTDGHCSKILRRCLCTKPC

SEQ ID NO: 46 - protein, 48 aa, artificial
  HXP34  + N-terminal alanine
ARECKTESQHHSFPCITKPPCRKACISEKFTDGHCSKILRRCLCTKPC

SEQ ID NO: 47 - protein, 48 aa, artificial
  HXP35  + N-terminal alanine
ARECKTESDTYRGVCITKPPCRKACISEKFTDGHCSKILRRCLCTKPC

(19) United States
(12) Patent Application Publication
(10) Pub. No.: US 2018/0193412 A1
(43) Pub. Date: Jul. 12, 2018

(54) Method of In Vivo Treatment

(71) Applicant: Hexima Limited, Bundoora (AU)

(72) Inventors: Nicole Louise Van der Weerden, Coburg (AU); Marilyn Anne Anderson, Keilor (AU)

(73) Assignee: Hexima Limited, Bundoora (AU)

(21) Appl. No.: 15/833,171

(22) Filed: Dec. 6, 2017

Related U.S. Application Data
(63) Continuation of application No. PCT/AU2015/050294, filed on May 29, 2015.

(51) Int. Cl. A61K 38/16; A61P 31/10; A61P 31/04; A61K 45/06; A61L 31/10

(52) U.S. Cl. CPC A61K 38/168; A61P 31/10; A61P 31/04; A61L 2300/252; A61L 31/10; A61L 2300/404; A61K 45/06

(57) ABSTRACT

The present disclosure teaches the treatment or prophylaxis of infection by a microorganism including a fungus or bacterium which is infecting or colonizing an in vivo tissue, surface or membrane. The method comprising administering to the subject with the infection or directly to the site of infection a plant-derived defensin or a functional variant or derivative thereof.

### BACKGROUND

### Field

The present disclosure relates generally to the control of in vivo microbial infection in humans and animals. Agents and natural and synthetic formulations and extracts useful for the control of microbial infection of non-external tissues, surfaces and membranes are also encompassed by the subject disclosure.

### Description of Related Art

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Microbial infection can lead to significant health issues in humans and animals. Examples include fungal and bacterial infection of mucosal tissue, respiratory surfaces, wounds and deep tissue.

Although antimicrobial agents including antibiotics and chemical microbicides have been successful in human and veterinary medicine, there is a range of environmental and regulatory concerns with the continued use of these microbicides for a host of reasons including the widespread development of resistance. There is clearly a need to develop alternative mechanisms of controlling infection in humans and animals by microbial pathogens or at least to complement existing antimicrobial agents.

The most prevelant human fungal pathogen is Candida. Candida is a commensal organism and part of the normal flora for 30-50% of the population but can cause disease in patients who are immunocompromised or whose natural microflora is disrupted such as through the use of antibiotics. Several species of Candida can cause infections in humans including Candida albicans, C. glabrata, C parasilosis, C. tropicalis and C. krusei. Common Candida infections include infections of the mucosal membranes such as oral and vaginal thrush. Oral candidiasis (oropharyngeal) is one of the most common infections observed in those suffering HIV (Singh et al. (2014) Journal of Oral and Maxillofacial Pathology 18(Suppl):S81-S85). More serious Candida infections include systemic blood stream infections (Candidemia) and biofilms on implanted medical devices. Candidemia is responsible for between 2 and 8 of every 1000 hospital intensive care admissions and has a 30-day mortality rate of 30%. Candidemia is characterised by spreading of Candida cells to the entire body creating abscesses in almost all vital organs, inducing their failure and leading to a morbidity rate of 50%. Antifungal agents belonging to the polyene, azole and echinocandin family have been used to treat Candidiasis, however, all have unwanted side effects such as toxicity, drug-drug interactions and resistance. Candidiasis is commonly observed in animals and often caused by C. albicans. Although C. albicans is a normal inhabitant of animal species, it can be an opportunistic pathogen. Birds are most commonly affected. Superficial infections have been described in pigs and foals. Systemic candidiasis has been observed in cattle, calves, sheep and foals following prolonged exposure to antibiotics.

Another serious fungal infection is aspergillosis. Aspergillosis comprises a large spectrum of fungal diseases caused by Aspergillus that primarily affect the lungs, although other organ systems can be affected. Clinical manifestations of lung aspergillosis are allergic bronchopulmonary aspergillosis, chronic necrotising aspergillosis, aspergilloma and the most severe, invasive aspergillosis (IA). IA is the most common filamentous fungal infection in immunocompromised patients (Patterson et al. (2000) Medicine 79(4):250-60). The genus Aspergillus includes over 185 species, which are ubiquitous in nature, especially common in soil and decaying vegetation. The most common pathogens that cause disease in humans are A. fumigatus, A. flavus, A. terreus, A. niger and A tubingensis. Reports have shown that attributable mortality of IA exceeds 80% (Fisher et al, (1981) The American Journal of Medicine 71(4):571-7). Current treatments involve members of polyene and azole family, including amphotericin b and isavuconazole respectively. These treatments display unwanted side effects such as toxicity, drug-drug interactions and for isavuconazole, the mortality rate remains at 18%. Aspergillosis is also a common fungal infection observed in animals. It is caused by several Aspergillus species such as A. fumigatus, A. terreus, A. niger, A. nidulans, A. viridnutans, A. flavus and A. felis. Animals that are affected include birds, horses, dogs, cats and cattle.

Cryptococcosis is another infectious disease caused by fungal pathogens. The manifestation may range from asymptomatic to mild bronchopneumonia to life-threatening infections of the central nervous system (CNS; Mitchell & Perfect (1995) Clinical Microbiology Reviews 8(4):515-48). Furthermore, cryptococcal meningitis is one of the most widely observed infections in patients suffering from AIDS (Vibhagool et al. (2003) Clinical Infectious Diseases 36(10):1329-31). The major causative agents of cryptococcosis are from the Cryptococcus family, generally C. neoformans and C. gatti. The most serious type of cryptococcal disease arises from uncontrolled pulmonary cryptococcosis, which progresses to cryptococcal meningitis. This progression tends only to occur in patients that are immunosuppressed. The most common treatment for cryptococcal meningitis is amphotericin B in combination with flucytosine. Early appropriate treatment has reduced the mortality rate from 14-25% to 6%, however, toxic side effects from amphotericin B are common including hypotension, anorexia, vomiting, headache and multiple organ damage. Cryptococcosis is also observed in animals. It is most common in cats but has also been reported in dogs, cattle, horses, sheep, goats, birds and wild animals. Transmission occurs via inhalation of spores or by contamination of wounds.

Penicilliosis is another common opportunistic infection in patients with HIV and 9.36% of patients with HIV develop Penicilliosis. The causative pathogen of penicilliosis is Penicillium marneffei (also known as Talaromyces marneffei). AIDS patients suffering from P. marneffei infection display symptoms of fever, anaemia, weight loss, lymphadenopathy, respiratory signs and skin lesions. The mortality for those that do not undergo treatment is at least 75% (Supparatpinyo et al. (1996) Lancet 344(8915):110-3).

Murcomycoses are the second most frequent mold infections observed in immunosuppressed patients. Most mucormycoses are life threatening and the most common presentation is severe infection of the sinuses, which may extend to the brain. Infectious agents belong to the order Mucorales with the most recognized causative agent belonging to the Genus Rhizopus (Rhizopus oryzae). However, additional mucormycosis causing species have been identified including Apophysomyces elegans, Cunninghamella bertholletiae, Saksenaea vasiformis, Rhizomucor pusillus, Syncephalostrum racemosum, Cokeromyces recurvatus, Actinomucor elegans (Gomes et al. (2011) Clinical Microbiology Reviews 24(2):411-45). The mortality rates are very high between 40°/h and 85%. The cutaneous forms of the infection represent the lowest mortality rate at 15% with the disseminated disease carrying a mortality rate approaching 100%. Treatments currently include amphotericin B, surgical procedures and azole treatments such as posaconazole.

Pythiosis is an emerging, life threatening infectious disease. It commonly affects horses and has been found in other animals such as dogs, cats and cattle causing granulomatous infections in the skin, intestines and arteries (Wanachiwanawin et al. (2004) Vaccine 22(27-28):3613-21). The first report of a human infection was reported in Thailand in 1985 and has since been reported in tropical and subtropical countries. The disease can manifest as a localized form, including eye infections, corneal ulcers with cutaneous or subcutaneous involvement. However, it can also present as a systemic or vascular infection and this is usually the most severe (Thianprasit et al. (1996) Current Topics in Medical Mycology 7(1):43-54). Clinical features are limb ischemia and gangrene and if the infection reaches a main artery, the patient will often die from systemic pythiosis. The major pathogen is Pythium insidiosum, a fungus-like aquatic organism from the phylum Oomycota. Antifungal treatments such as amphotericin B and various azoles display poor efficacy for systemic and vascular phythiosis and the most effective treatment is considered to be amputation of the site of infection.

Mycotic keratitis has been reported in many parts of the world, particularly tropical areas. There are two major forms, one caused by filamentous fungi such as Fusarium and Aspergillus and another form caused by yeast-like pathogens such as Candida. Traumatic injury is a major predisposing factor for keratitis caused by filamentous fungi. The major filamentous fungal species involved are F. solani, F. oxysporum, A. fumigatus and A flavus (Thomas (2003) Eye (London, England) 17(8):853-62). Topical natamycin and amphotericin B are used as a first line of treatment, however if deep lesions are present oral ketoconazole, itraconazole or fluconazole may be administered with reasonable response rates (Thomas (2003) supra). In cases where medical treatments fail, surgical intervention may be necessary.

Histoplasmosis is a fungal infection that contracted after inhalation of the spores of the thermally dimorphic fungus Histoplasmosis capsulatum. This pathogen is found in North America, South America, Africa and Asia. There are several types of histoplasmosis, with the mildest form producing little or any symptoms at all. However, infants or those with compromised immune systems may develop progressive disseminated histoplasmosis, which can be fatal if left untreated (Wheat et al. (1990) Medicine 69(6):361-74). Symptoms include fever, chills, headache, muscle aches and a dry cough. For severe infections, amphotericin B is recommended for 1-2 weeks followed by itraconazole. Amphotericin B displays very high, some lethal, levels of toxicity.

Histoplasmosis has also been described in many animal species, however infection is rare in dogs and cats. Infection usually arises following aerosol exposure to the respiratory tract.

Blastomycosis is caused by the dimorphic fungus Blastomyces dermatitidis. Infection is most common among dogs and cats but it has also been observed in horses, ferrets, wolves, deer, lions and dolphins. It is limited to North America and transmission is thought to be via inhalation of aerosolised conidia.

Plant defensins are small (45-54 amino acids), basic proteins with four to five disulfide bonds (Janssen et al. (2003) Biochemistry 42(27):8214-8222). They share a common disulfide bonding pattern and a common structural fold, in which a triple-stranded, antiparallel β-sheet is tethered to an α-helix by three disulfide bonds, forming a cysteine-stabilized αβ motif. A fourth disulfide bond also joins the N- and C-termini leading to an extremely stable structure. A variety of functions has been attributed to defensins, including anti-bacterial activity, protein synthesis inhibition and α-amylase and protease inhibition (Colilla et al. (1990) FEBS Lett 270(1-2)191-194; Bloch and Richardson (1991) FEBS Lett 279(1):101-104), generally in the context of plants.

The structure of defensins consists of seven ‘loops’, defined as the regions between cysteine residues. Loop 1 encompasses the first β-strand (1A) as well as most of the flexible region that connects this β-strand to the α-helix (1B) between the first two invariant cysteine residues. Loops 2, 3 and the beginning of 4 (4A) make up the α-helix, while the remaining loops (4B-7) make up β-strands 2 and 3 and the flexible region that connects them (β-hairpin region) (van der Weerden et al. (2013) Cell Mol Life Sci 70 (19): 3545-3570). Loop 5 of the plant defensins is known to be essential for antifungal activity and an important determinant for mechanism of action of these proteins (Sagaram et al., (2011) PLoS One 6.4: e18550).

Plant defensins generally share eight completely conserved cysteine residues. These residues are commonly referred to as “invariant cysteine residues”, as their presence, location and connectivity are conserved amongst defensins. Based on sequence similarity, plant defensins can be categorized into different groups. Within each group, sequence homology is relatively high whereas inter-group amino acid similarity is low (van der Weerden et al. (2013) Fungal Biol Rev 26:121-131). Plant defensins belonging to different groups generally have different biological activities or different mechanisms of action for the same biological activity.

There are two major classes of plant defensins. Class I defensins consist of an endoplasmic reticulum (ER) signal sequence followed by a mature defensin domain. Class II defensins are produced as larger precursors with C-terminal pro-domains or pro-peptides (CTPPs) of about 33 amino acids. Most of the Class II defensins identified to date have been found in Solanaceous plant species.

There is a need to develop protocols to more effectively manage microbial infection in humans and animals. Whilst some defensins have antifungal properties, their activities across different fungal pathogens vary significantly and the majority of demonstrated activity has been toward plant fungal pathogens.

### SUMMARY

Amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present disclosure teaches a method for inhibiting infection by a microorganism in in vivo tissue in a subject the method comprising contacting the in vivo tissue with an effective amount of a plant defensin or a functional natural or synthetic derivative or variant thereof, the plant defensin, derivative or variant selected from the list consisting of SEQ ID NO:1 through 47 or a plant defensin having at least about 80% amino acid sequence similarity to any one of SEQ ID NO:1 through 47 after optimal alignment for a time and under conditions sufficient to eradicate or otherwise control microbial growth or colonization. Despite evolving to target plant pathogens, the plant defensins defined herein have potent activity against microorganism with no or medically acceptable minimal off target activity on mammalian cells. More surprisingly, many of the defensins have a microbicidal activity on contact.

The defensins defined by SEQ ID NO:1 through 47 are defined in Tables 1 and 2. The present invention encompasses the treatment of any internal surface tissue or membrane which is not external skin, hair or nails. In an embodiment, the defensin is defined by the consensus amino acid sequence SEQ ID NO:24 or a functional natural or synthetic derivative or variant thereof which includes a defensin having at least 80% similarity to SEQ ID NO: 24 after optimal alignment wherein SEQ ID NO:24 has an optional N-terminal alanine residue. Examples include SEQ ID NO: 1, SEQ ID NO:2 and SEQ ID NO:3 and their variants with an N-terminal alanine residue (SEQ ID NO: 25 though 27, respectively). Reference to “external skin” does not exclude subcutaneous layers of external skin or a surface wound.

Examples of microbial infection include infection by fungi and bacteria. These include infections by Candida such as of mucosal membranes (e.g. thrush), the gastrointestinal tract and the blood stream, infections by Cryptococcus such as meningitis, infections by Aspergillus such as respiratory infections, subcutaneous infections such as mucorymycosis, bacterial gastroenteritis, respiratory infection, wound infection and infection leading to a sexually transmitted disease. Also included are infections by E. coli, such as diarrhoea and urinary tract infections and by Bacillus spp. such as ear infections, meningitis, urinary tract infections and septicaemia.

The defensins contemplated for use in the treatment protocol are defined by the amino acid sequence selected from SEQ ID NO:1 through 47 listed in Table 1 and further characterized in Table 2 as well as a defensin having at least 80% similarity to any one of the SEQ ID NO:1 through 47 after optimal alignment. For the sake of brevity, reference to “SEQ ID NO:1 through 47” hereinafter includes defensins having at least 80% similarity to any one of SEQ ID NO:1 through 47. In an embodiment, the defensin is a permeabilizing defensin or a functional natural or synthetic derivative or variant thereof. Examples of synthetic variants include where a Loop1B from a Class I defensin replaces the Loop1B from the Solanaceous Class II defensin. These include HXP4, HXP34 and EIXP35. Other variants or derivatives include a defensin listed in Table 2 or having at least 80% similarity to a defensin listed in Table 2 wherein the defensin comprises an alanine residue at its N-terminus (i.e. SEQ ID NO:25 through 47). The addition of an alanine at the N-terminus of a defensin allows the peptide to be produced recombinantly in the Pichia pastoris expression system without the need for the STE13 protease site. The STE13 site normally allows for efficient processing of the α-mating factor secretion signal by KEX2. However, under high expression loads the STE13 protease cleavage can be inefficient leading to Glu-Ala repeats remaining on the N-terminus of the peptide. The additional negative charge conferred by these repeats can be detrimental to the activity of plant defensins. The STE13 protease site can be replaced with an alanine to prevent incomplete processing (Cabral et al., (2003) Protein Express Purif 31(1):115-122). The presence of an N-terminal alanine can also decrease the ability of the defensin to lyse red blood cells (WO2011/16074).

In an embodiment, a defensin listed in Table 2 (SEQ ID NO:1 through 47) is modified to enhance the stability of the peptide. In a further embodiment this is achieved by replacing amino acids that are susceptible to deamidation such as asparagine and glutamine, or amino acids that are susceptible to isomerization such as aspartic acid, with amino acids that are not susceptible to modifications. In a particular embodiment, the defensins HXL008, HXL035 or HXL036 are modified at positions 18, 36 or 42.

In an embodiment, a defensin listed in Table 2 (SEQ ID NO:1 through 47) is modified to increase the positive charge of the peptide. Positive charge is known to be important for the activity of antimicrobial peptides, including plant defensins (Sagaram et al., (2011) PLoS One 6.4: e18550). In an embodiment the increase in positive charge is achieved by replacement of a negatively charged residue such as glutamatic acid or aspartic acid with a neutral amino acid. In a preferred embodiment the neutral amino acid is an alanine or a glycine. In another embodiment, the increase in positive charge is achieved by replacing a neutral amino acid with a positively charged residue such as lysine or arginine.

In an embodiment, the treatment includes a defensin in combination with a non-defensin peptide, a proteinase inhibitor, another defensin or a proteinaceous or non-proteinaceous (i.e. chemical) microbicide including an antibiotic.

In an embodiment, the defensin is provided by any means including by oral, inhalation, intravenous, sublingual, intraperitoneal, rectal or subcutaneous administration. Alternatively, it is topically applied to an internal tissue, surface or membrane or to a wound. The defensin can also be administered via a slow release patch or implant (e.g. a subcutaneous implant). The defensins can also be coated on the surface of medical devices such as catheters and implants or condoms or other sheaths.

Further taught herein is a formulation or extract comprising a plant defensin selected from SEQ ID NO:1 through 47. The formulation or extract may further comprise another active agent or a combination of formulations or extracts wherein at least one formulation or extract comprises a defensin as defined herein which are admixed prior to use or used sequentially in either order. The plant defensins or extracts comprising same as defined herein may be used such as in the form of systemic or local formulations, coatings, gels, ointments, cream, spray, foam, capsule, tablet, oral formulations, inhalable or atomized formulations and the like including herbal formulations and plant extracts.

Enabled herein is a use of a plant defensin as defined by SEQ ID NO:1 through 47 in the manufacture of a medicament for the treatment or prophylaxis of microbial infection of in vivo tissue in a subject. Also taught herein is a plant defensin as defined by SEQ ID NO:1 through 47 for use in the treatment or prophylaxis of microbial infection of in vivo tissue in a subject. Further taught herein is a plant defensin as defined by SEQ ID NO:1 through 47 when used in the treatment of microbial infection of in vivo tissue in a subject. In an embodiment, the defensin has no adverse activity or medically acceptable minimal activity against mammalian cells. Activity against mammalian cells will increase the likelihood of dermal irritation and other side effects.

As indicated above, the defensin may also be a functional natural or synthetic derivative or variant of a defensin as defined by any one of SEQ ID NO: 1 through 47. The subject may be a human or non-human animal subject.

Further contemplated herein is an isolated microorganism engineered to express a defensin as defined herein for use in the manufacture of compositions comprising the microorganisms. In an embodiment, the microorganism is a yeast such as but not limited to Pichia. Such compositions are useful in the treatment of humans and animals such as in the form of a probiotic. Alternatively, the defensin is provided as a cell extract including a plant extract or microbial extract.

A kit in compartmental form comprising a plant defensin or a functional natural or synthetic derivative or variant thereof, the plant defensin selected from the list consisting of SEQ ID NO: 1 through 47, is also taught herein. In an optional embodiment, another compartment comprises a second active agent and optionally separably in a further compartment or together in an existing compartment, a pharmaceutically or veterinarily acceptable diluent, carrier or excipient. In an embodiment, the defensin is defined by the consensus amino acid sequence set forth in SEQ ID NO: 24. Examples include HXL008 (SEQ ID NO: 1), HXL035 (SEQ ID NO: 2), HXL036 (SEQ ID NO: 3) and the N-terminal alanine forms, SEQ ID NO: 25 though 27, respectively.

In an embodiment, a defensin contemplated for use herein may or may not comprise an N-terminal alanine residue. This is particularly the case with some recombinant defensins which comprise the N-terminal alanine residue. Encompassed by the definition of a “defensin” herein is any of SEQ ID NO:1 though 23 with an N-terminal alanine. These are represented as SEQ ID NO:25 through 47. The consensus amino acid sequence, SEQ ID NO: 24, has an optional N-terminal alanine residue.

The addition of an alanine at the N-terminal of a defensin allows the peptide to be produced predominantly in Pichia pastoris expression system without need for the STE13 protease site. (Cabral et al. (2003) Protein Express Purif 31(1):115-122). The presence of the N-terminal alanine can also decrease the ability of a defensin to lysine red blood cells.

In an embodiment, a defensin listed in Table 2 (SEQ ID NO: 1 through 47) is modified to enhance the stability of the peptide. In a further embodiment, this is achieved by replacing amino acids that are susceptible to deamidation such as asparagine and glutamine, or amino acids that are susceptible to isomerization such as aspartic acid, with amino acids that are not susceptible to modifications. In a particular embodiment, the defensins HXL008, HXL035 or HXL036 are modified at positions 18, 36 or 42.

In an embodiment, a defensin listed in Table 2 (SEQ ID NO:1 through 47) is modified to increase the positive charge of the peptide. Positive charge is known to be important for the activity of antimicrobial peptides, including plant defensins. In an embodiment the increase in positive charge is achieved by replacement of a negatively charged residue such as glutamatic acid or aspartic acid with a neutral amino acid. In an embodiment, the neutral amino acid is an alanine or a glycine. In another embodiment, the increase in positive charge is achieved by replacing a neutral amino acid with a positively charged residue such as lysine or arginine

Conservative amino acid changes are also contemplated herein.

TABLE 1

| SEQUENCE | |
|---|---|
| ID NO: | DESCRIPTION |
| 1 | Amino acid sequence of HXL008 protein |
| 2 | Amino acid sequence of HXL035 protein |
| 3 | Amino acid sequence of HXL036 protein |
| 4 | Amino acid sequence of HXL001 protein |
| 5 | Amino acid sequence of HXL002 protein |
| 6 | Amino acid sequence of HXL003 protein |
| 7 | Amino acid sequence of HXL004 protein |
| 8 | Amino acid sequence of HXL005 protein |
| 9 | Amino acid sequence of HXL009 protein |
| 10 | Amino acid sequence of HXL012 protein |
| 11 | Amino acid sequence of HXL013 protein |
| 12 | Amino acid sequence of HXL015 protein |
| 13 | Amino acid sequence of HXL032 protein |
| 14 | Amino acid sequence of HXL033 protein |
| 15 | Amino acid sequence of HXL034 protein |
| 16 | Amino acid sequence of NsD1 protein |
| 17 | Amino acid sequence of NsD2 protein |
| 18 | Amino acid sequence of NaD1 protein |
| 19 | Amino acid sequence of NoD173 protein |
| 20 | Amino acid sequence of DmAMP1 protein |
| 21 | Amino acid sequence of HXP4 protein |
| 22 | Amino acid sequence of HXP34 protein |
| 23 | Amino acid sequence of HXP35 protein |
| 24 | Consensus sequence |
| 25 | Amino acid sequence of HXL008 protein + N-terminal |
| | alanine |
| 26 | Amino acid sequence of HXL035 protein + N-terminal |
| | alanine |
| 27 | Amino acid sequence of HXL036 protein + N-terminal |
| | alanine |
| 28 | Amino acid sequence of HXL001 protein + N-terminal |
| | alanine |
| 29 | Amino acid sequence of HXL002 protein + N-terminal |
| | alanine |
| 30 | Amino acid sequence of HXL003 protein + N-terminal |
| | alanine |
| 31 | Amino acid sequence of HXL004 protein + N-terminal |
| | alanine |
| 32 | Amino acid sequence of HXL005 protein + N-terminal |
| | alanine |
| 33 | Amino acid sequence of HXL009 protein + N-terminal |
| | alanine |
| 34 | Amino acid sequence of HXL012 protein + N-terminal |
| | alanine |
| 35 | Amino acid sequence of HXL013 protein + N-terminal |
| | alanine |
| 36 | Amino acid sequence of HXL015 protein + N-terminal |
| | alanine |
| 37 | Amino acid sequence of HXL032 protein + N-terminal |
| | alanine |
| 38 | Amino acid sequence of HXL033 protein + N-terminal |
| | alanine |
| 39 | Amino acid sequence of HXL034 protein + N-terminal |
| | alanine |
| 40 | Amino acid sequence of NsD1 protein + N-terminal alanine |
| 41 | Amino acid sequence of NsD2 protein + N-terminal alanine |
| 42 | Amino acid sequence of NaD1 protein + N-terminal alanine |
| 43 | Amino acid sequence of NoD173 protein + N-terminal |
| | alanine |
| 44 | Amino acid sequence of DmAMP1 protein + N-terminal |
| | alanine |
| 45 | Amino acid sequence of HXP4 protein + N-terminal alanine |
| 46 | Amino acid sequence of HXP34 protein + N-terminal |
| | alanine |
| 47 | Amino acid sequence of HXP35 protein + N-terminal |
| | alanine |

Amino acid sequences for HXL proteins are recited in U.S. Pat. No. 6,911,577 and related patent family members.

TABLE 2
Examples of plant defensins

| | | Type | |
|---|---|---|---|
| | | (Class I, | |
| | | Class II | |
| | | or | |
| Peptide | Source | variant) | Reference |
| HXL008 | Picramnia | Class I | SEQ ID NO: 1 |
| | pentandra | | |
| HXL035 | Picramnia | Class I | SEQ ID NO: 2 |
| | pentandra | | |
| HXL036 | Picramnia | Class I | SEQ ID NO: 3 |
| | pentandra | | |
| HXL001 | Zea mays | Class I | SEQ ID NO: 4 |
| HXL002 | Triticum aestivum | Class I | SEQ ID NO: 5 |
| HXL003 | Triticum aestivum | Class I | SEQ ID NO: 6 |
| HXL004 | Nicotiana | Class I | SEQ ID NO: 7 |
| | benthamiana | | |
| HXL005 | Taraxcum kok- | Class I | SEQ ID NO: 8 |
| | saghyz | | |
| HXL009 | Zea mays | Class I | SEQ ID NO: 9 |
| HXL012 | Amaranthus | Class I | SEQ ID NO: 10 |
| | retroflexus | | |
| HXL013 | Glycine max | Class I | SEQ ID NO: 11 |
| HXL015 | Oryza sativa | Class I | SEQ ID NO: 12 |
| HXL032 | Triticum aestivum | Class I | SEQ ID NO: 13 |
| HXL033 | Parthenium | Class I | SEQ ID NO: 14 |
| | argentatum | | |
| HXL034 | Nicotiana | Class I | SEQ ID NO: 15 |
| | benthamiana | | |
| NsD1 | Nicotiana | Class II | SEQ ID NO: 16 |
| | suaveolens | | |
| NsD2 | Nicotiana | Class II | SEQ ID NO: 17 |
| | suaveolens | | |
| NaD1 | Nicotiana alata | Class II | SEQ ID NO: 18 |
| NoD173 | Nicotiana | Class II | SEQ ID NO: 19 |
| | occidentalis spp | | |
| | obliqua | | |
| DmAMP1 | Dahlia merckii | Class I | SEQ ID NO: 20 |
| HXP4 | Artificial | Variant | SEQ ID NO: 21 |
| HXP34 | Artificial | Variant | SEQ ID NO: 22 |
| HXP35 | Artificial | Variant | SEQ ID NO: 23 |
| Consensus | | | SEQ ID NO: 24 |
| HXL008 + Ala | Artificial | Variant | SEQ ID NO: 25 |
| HXL035 + Ala | Artificial | Variant | SEQ ID NO: 26 |
| HXL036 + Ala | Artificial | Variant | SEQ ID NO: 27 |
| HXL001 + Ala | Artificial | Variant | SEQ ID NO: 28 |
| HXL002 + Ala | Artificial | Variant | SEQ ID NO: 29 |
| HXL003 + Ala | Artificial | Variant | SEQ ID NO: 30 |
| HXL004 + Ala | Artificial | Variant | SEQ ID NO: 31 |
| HXL005 + Ala | Artificial | Variant | SEQ ID NO: 32 |
| HXL009 + Ala | Artificial | Variant | SEQ ID NO: 33 |
| HXL012 + Ala | Artificial | Variant | SEQ ID NO: 34 |
| HXL013 + Ala | Artificial | Variant | SEQ ID NO: 35 |
| HXL015 + Ala | Artificial | Variant | SEQ ID NO: 36 |
| HXL032 + Ala | Artificial | Variant | SEQ ID NO: 37 |
| HXL033 + Ala | Artificial | Variant | SEQ ID NO: 38 |
| HXL034 + Ala | Artificial | Variant | SEQ ID NO: 39 |
| NsD1 + Ala | Artificial | Variant | SEQ ID NO: 40 |
| NsD2 + Ala | Artificial | Variant | SEQ ID NO: 41 |
| NaD1 + Ala | Artificial | Variant | SEQ ID NO: 42 |
| NoD173 + Ala | Artificial | Variant | SEQ ID NO: 43 |
| DmAMP1 + | Artificial | Variant | SEQ ID NO: 44 |
| Ala | | | |
| HXP4 + Ala | Artificial | Variant | SEQ ID NO: 45 |
| HXP34 + Ala | Artificial | Variant | SEQ ID NO: 46 |
| HXP35 + Ala | Artificial | Variant | SEQ ID NO: 47 |

### BRIEF DESCRIPTION OF THE FIGURES

FIG. 1(A) is a representation of alignments of amino acids of the various defensins encompassed therein.

FIG. 1(B) is a representation of alignments of amino acids of the various defensins encompassed therein.

FIG. 1(C) is a representation of alignments of amino acids of the various defensins encompassed therein.

FIG. 1(D) is a representation of alignments of amino acids of the various defensins encompassed therein.

FIG. 1(E) is a representation of alignments of amino acids of the various defensins encompassed therein.

FIG. 2(A) is a graphical representations showing the effect of the plant defensins NaD1 (dashed line) and HXL008 (solid line) on the growth of clinical isolate 14-01 of Trichophyton rubrum in vitro. Fungal growth was measured by the increase in optical density at 595 nm (A595) achieved 72 hours after inoculation of the growth medium and is plotted as a percentage of growth relative to a no-protein control (vertical axis) versus protein concentration (μg/mL, horizontal axis).

FIG. 2(B) is a graphical representation showing the effect of the plant defensins NaD1 (dashed line) and HXL008 (solid line) on the growth of clinical isolate 14-02 of Trichophyton rubrum in vitro. Fungal growth was measured by the increase in optical density at 595 nm (A595) achieved 72 hours after inoculation of the growth medium and is plotted as a percentage of growth relative to a no-protein control (vertical axis) versus protein concentration (μg/mL, horizontal axis).

FIG. 2(C) is a graphical representation showing the effect of the plant defensins NaD1 (dashed line) and HXL008 (solid line) on the growth of clinical isolate 14-03 of Trichophyton rubrum in vitro. Fungal growth was measured by the increase in optical density at 595 nm (A595) achieved 72 hours after inoculation of the growth medium and is plotted as a percentage of growth relative to a no-protein control (vertical axis) versus protein concentration (μg/mL, horizontal axis).

FIG. 2(D) is a graphical representation showing the effect of the plant defensins NaD1 (dashed line) and HXL008 (solid line) on the growth of clinical isolate 13-04 of Trichophyton rubrum in vitro. Fungal growth was measured by the increase in optical density at 595 nm (A595) achieved 72 hours after inoculation of the growth medium and is plotted as a percentage of growth relative to a no-protein control (vertical axis) versus protein concentration (μg/mL, horizontal axis).

FIG. 3(A) is a photographic representations of surviving colonies of T. rubrum clinical isolate 14-01 grown on agar plates after treatment with 100 μM HXL008 for 72 h.

FIG. 3(B) is a photographic representation of surviving colonies of T. rubrum clinical isolate 14-02 grown on agar plates after treatment with 100 μM HXL008 for 72 h.

FIG. 3(C) is a photographic representation of surviving colonies of T. rubrum clinical isolate 14-03 grown on agar plates after treatment with 100 μM HXL008 for 72 h.

FIG. 3(D) is a photographic representation of surviving colonies of T. rubrum clinical isolate 13-04 grown on agar plates after treatment with 100 μM HXL008 for 72 h.

FIG. 3(E) are photographic representations of T. Rubrum, which was not treated with any HXL008 (left panels) after 24 hours and 14 days respectively, T. Rubrum, which was treated with 10 μg/mL HXL008 (center panels) after 24 hours, 14 days and 25 days, respectively, T. Rubrum, (right panels) which was treated with 50 μg/mL HXL008 (center panels) after 24 hours, 14 days and 25 days, respectively.

FIG. 4 is a representation of an amino acid alignment of HXL008, HXL035 and HXL036 and a consensus sequence of these three sequences. Identical amino acids are highlighted in black conserved amino acids are highlighted in grey.

### DETAILED DESCRIPTION

Throughout this specification, unless the context requires otherwise, the word “comprise” or variations such as “comprises” or “comprising”, will be understood to imply the inclusion of a stated element or integer or method step or group of elements or integers or method steps but not the exclusion of any element or integer or method step or group of elements or integers or method steps.

As used in the subject specification, the singular forms “a”, “an” and “the” include plural aspects unless the context clearly dictates otherwise. Thus, for example, reference to “a defensin” includes a single defensin, as well as two or more defensins; reference to “an agent” includes single agent, as well as two or more agents; reference to “the disclosure” includes a single and multiple aspects taught by the disclosure; and so forth. Aspects taught and enabled herein are encompassed by the term “invention”. All such aspects are enabled within the width of the present invention. Any variants and derivatives contemplated herein are encompassed by “forms” of the invention.

Reference to a “defensin” means one or more of the following plant defensins which retains antimicrobial activity.

(i) a defensin having the consensus amino acid sequence as set forth in SEQ NO:24;
(ii) a defensin selected from the group consisting of HXL008 (SEQ ID NO:1), HXL035 (SEQ ID NO:2) and HXL036 (SEQ ID NO:3);
(iii) a defensin selected from the group consisting of HXL001 (SEQ ID NO:4), HXL002 (SEQ ID NO:5), HXP35 (SEQ ID NO:3), HXL003 (SEQ ID NO:6), HXL004 (SEQ ID NO:7), HXL005 (SEQ ID NO:8), HXL009 (SEQ ID NO:9), HXL012 (SEQ ID NO:10), HXL013 (SEQ NO:11), HXL015 (SEQ ID NO:12), HXL032 (SEQ NO:13), HXL033 (SEQ ID NO:14), HXL034 (SEQ ID NO:15), NsD1 (SEQ ID NO:16), NsD2 (SEQ ID NO:17), NaD1 (SEQ ID NO:18), NoD173 (SEQ ID NO:19), DmAMP1 (SEQ ID NO:20), HXP4 (SEQ ID NO:21), HXP34 (SEQ ID NO:22) and HXP35 (SEQ ID NO:23).
(iv) a functional naturally occurring or synthetic derivative or variant of any one of SEQ ID NO:1 through 47;
(v) a defensin having at least 80% similarity to any one of SEQ ID NO:1 through 47 after optimal alignment;
(vi) any one of SEQ ID NO:1 through 47 comprising a N-terminal alanine residue (i.e. SEQ ID NO:25 through 47); and/or
(vii) a defensin having at least 80% similarity to any one of SEQ ID NO:1 through 3 or after optimal alignment.

In an embodiment, the defensin is defined by SEQ ID NO: 1 though 3 or contains an N-terminal alanine residue (SEQ ID NO: 25 through 27 respectively). For convenience these defensins are encompassed within the consensus amino acid sequence set forth in SEQ ID No: 24.

Reference hereinafter to a “defensin” or a defensin “defined herein”, means a defensin as listed in paragraphs (i) through (vii) above. The different defensins encompassed by (i) through (vii) represent different forms of the subject invention.

When the defensin herein is used in combination with a microbicidic agent and that microbicidic agent is a defensin, then the second defensin may be any defensin.

A protocol is described herein which is used to facilitate management of microbial infection or colonization at particular in vivo anatomical sites or regions in a human or non-human animal subject. The protocol comprises the use of a plant defensin or a functional natural or synthetic derivative or variant thereof to inhibit or otherwise control the growth or colonization of a microorganism on or in in vivo tissue. The present protocol excludes the treatment of skin, hair and nails. However, reference to skin does not exclude subcutaneous infection of skin or a surface wound. In an embodiment, the defensin has no adverse activity or medically acceptable minimal activity against mammalian cells. In an optional embodiment, the defensin is used in synergistic combination with another antimicrobial agent. The latter agent includes a non-defensin peptide, a proteinase inhibitor, another defensin and a proteinaceous or non-proteinaceous (chemical) agent with antimicrobial properties including an antibiotic.

Examples of microbial infection include infection by fungi and bacteria. These include aspergillosis, infection by Candida such as of mucosal membranes (e.g. thrush), systemic candidiasis, cryptococcosis, subcutaneous infections such as mucorymycosis, bacterial gastroenteritis, respiratory infection, wound infection and an infection leading to a sexually transmitted disease.

Enabled herein is a method for inhibiting infection by a microorganism on or in in vivo tissue in a subject, the method comprising contacting the microorganism or tissue comprising the microorganism with an effective amount of a plant defensin or a functional natural or synthetic derivative or variant thereof. In an embodiment, the in vivo tissue is a mucosal or epithelial internal surface or tissue or a subcutaneous layer of skin. Hence, taught herein is a method for inhibiting growth or colonization of a microorganism on or in in vivo mucosal or epithelial tissue or a subcutaneous layer, the method comprising contacting the tissue or microorganism with an effective amount of a plant defensin or a functional natural or synthetic variant or derivative thereof. Generally, the contact is for a time and under conditions sufficient to inhibit growth of the microorganism. The defensins can also be coated on the surface of medical devices such as catheters and implants or condoms or other sheaths.

“Microbial inhibition” includes both microbiocidic and microbistatic activity, as measured by reduction of microbial biomass (or loss of viability) compared to a control. Microbial growth can be measured by many different methods known in the art depending on the microorganism. For fungi, for example, a commonly used method of measuring growth of a filamentous fungus, entails germinating spores in a suitable growth medium, incubating for a time sufficient to achieve measurable growth, and measuring increased optical density in the culture after a specified incubation time. The optical density rises with increased growth. Typically, microbial growth or colonization is necessary for pathogenesis. Therefore, inhibition of pathogen growth provides a suitable indicator for protection from microbial disease, i.e. the greater the inhibition, the more effective the protection. Furthermore, the effectiveness of the microbicidal or microstatic activity can be determined by microscopic examination or culture techniques from a sample or amelioration of disease symptoms (e.g. fever, redness, tenderness etc.).

Reference to microorganism includes a fungus and a bacterium.

The treatment protocol includes prophylaxis (i.e. prevention) of at risk subjects from infection. A subject “at risk” may be a subject in a particular location or demographic. Hence, “preventing infection” in the present context, means that the human or animal host is treated with the defensin so as to avoid microbial infection or disease symptoms associated therewith or exhibit reduced or minimized or less frequent microbial infection or disease symptoms associate therewith, that are the natural outcome of the host-microorganism interactions when compared to the host not exposed to the defensin. That is to say, microbial infection is prevented or reduced from causing disease and/or the associated disease symptoms. Infection and/or symptoms are reduced by at least about 10%, 20%, 30%, 40%, 50, 60%, 70% or 80% or greater as compared to a host not so treated with the protocol taught herein. The percentage reduction can be determined by any convenient means appropriate to the host and microorganism. The microorganism may be naturally occurring in the subject without being a “pathogen”. However, situations can arise where the microorganism multiplies to a level where it becomes pathogenic.

The action of the defensin is to inhibit microbial growth, replication, infection and/or maintenance, amongst other inhibitory activities and/or induce amelioration of symptoms of microbial infection when the level of microorganism or its location in the body (i.e. in the in vivo tissue, surface or membrane) results in a pathogenic outcome.

Enabled herein is a method for the treatment or prophylaxis of aspergillosis or a related condition in a subject, the method comprising contacting an infected site on the subject or administering to the subject a plant defensin or a functional natural or synthetic derivative or variant thereof for a time and under conditions sufficient to ameliorate the symptoms of aspergillosis.

In an embodiment, the microbial infection is infection by a species or strain of Candida.

Enabled herein is a method for the treatment or prophylaxis of Candida infection of a mucosal membrane in a subject, the method comprising contacting an infected site on the subject or administering to the subject a plant defensin or a functional natural or synthetic derivative or variant thereof for a time and under conditions sufficient to ameliorate the symptoms of infection.

In an embodiment, the Candida infection leads to thrush.

In an embodiment, the microbial infection is systemic candidiasis.

Hence, taught herein is a method for the treatment or prophylaxis of systemic candidiasis in a subject, the method comprising administering to the subject a plant defensin or a functional natural or synthetic derivative or variant thereof for a time and under conditions sufficient to ameliorate the symptoms of infection.

In an embodiment, the microbial infection is cryptococcosis.

Hence, taught herein is a method for the treatment or prophylaxis of cryptococcosis in a subject comprising contacting an infected site on the subject or administering to the subject a plant defensin or a functional natural or synthetic derivative or variant thereof for a time and under conditions sufficient to ameliorate the symptoms of infection.

In an embodiment, the microbial infection is mucormycosis or a related condition. According, enabled herein is a method for the treatment or prophylaxis of mucormycosis in a subject comprising contacting an infected site on the subject or administering to the subject a plant defensin or a functional natural or synthetic derivative or variant thereof for a time and under conditions sufficient to ameliorate the symptoms of infection.

In an embodiment, the microbial infection is a subcutaneous infection. Hence, taught herein is a method for the treatment or prophylaxis of infection of a subcutaneous skin layer in a subject, the method comprising contacting an infected site on the subject or administering to the subject a plant defensin or a functional natural or synthetic derivative or variant thereof for a time and wider conditions sufficient to ameliorate the symptoms of infection.

In an embodiment, the microbial infection is histoplasmosis. Hence, taught herein is a method for the treatment or prophylaxis of histoplasmosis in a subject comprising contacting an infected site on the subject or administering to the subject a plant defensin or a functional natural or synthetic derivative or variant thereof for a time and under conditions sufficient to ameliorate the symptoms of infection.

In an embodiment, the microbial infection leads to gastroenteritis.

Hence, the present specification is instructional for a method for the treatment or prophylaxis of microbial gastroenteritis in a subject comprising administering to the subject a plant defensin or a functional natural or synthetic derivative or variant thereof for a time and under conditions sufficient to ameliorate the symptoms of infection.

In an embodiment, the microbial infection is a respiratory infection.

Enabled herein is a method for the treatment or prophylaxis of respiratory infection in a subject, the method comprising administering to the subject a plant defensin or a functional natural or synthetic derivative or variant thereof for a time and under conditions sufficient to ameliorate the symptoms of infection.

In an embodiment, the microbial infection is a wound infection.

The subject specification teaches a method for the treatment or prophylaxis of a wound infection in or on a subject, the method comprising contacting the wound on the subject or administering to the subject a plant defensin or a functional natural or synthetic derivative or variant thereof for a time and under conditions sufficient to ameliorate the symptoms of infection.

In an embodiment, the microbial infection leads to a sexually transmitted disease.

Hence, enabled herein is a method for the treatment or prophylaxis of sexually transmitted disease in a subject, the method comprising contacting an infected site on the subject or administering to the subject a plant defensin or a functional natural or synthetic derivative or variant thereof for a time and under conditions sufficient to ameliorate the symptoms of infection.

In an embodiment, the defensin or its functional natural or synthetic derivative or variant is coated on to the surface of a medical device or condom. Examples of medical devices include catheters and implants. Any of the defensins defined herein may be used in these methods such as but not limited to a defensin defined by the consensus amino acid sequence SEQ ID NO:24 or a functional natural or synthetic derivative or variant thereof which includes a defensin having at least 80% similarity to SEQ ID NO: 24 after optimal alignment or wherein SEQ ID NO:24 has an optional N-terminal alanine residue. Examples include SEQ ID NO:1, SEQ ID NO:2 and SEQ ID NO:3 and variants thereof with an N-terminal alanine residue (SEQ ID NO:25 though 27, respectively).

By “contacting” includes exposure of the microorganism or tissue, surface or membrane or site comprising the microorganism to the defensin following administration or application to the human or animal subject. It also includes systemic, local, topical or parenteral administration to the subject or an infected site. Contact may be with a purified plant defensin or formulation comprising same, or a plant extract which comprises the defensin naturally or which has been engineered to produce the defensin. A formulation includes herbal formulations and pharmaceutical formulations. Reference to “contacting” includes the step of administering to a subject or an infected site on a subject.

Enabled herein is a formulation comprising a plant defensin or a functional natural or synthetic derivative or variant thereof for use in inhibiting infection by a microorganism on or in in vivo tissue in a human or animal subject.

Further enabled herein a use of a formulation comprising a plant defensin or a functional natural or synthetic derivative or variant thereof in the manufacture of a medicament for inhibiting infection by a microorganism on or in in vivo tissue in a human or animal subject.

Also enabled is a formulation comprising a plant defensin or a functional natural or synthetic derivative or variant thereof when used to inhibit infection by a microorganism on or in in vivo tissue in a human or animal subject.

In an embodiment, taught herein is a therapeutic kit comprising a compartment or in compartmental form wherein a compartment comprises a plant defensin or a functional natural or synthetic derivative or variant thereof. Second or further compartments may comprise other agents or excipients including other antimicrobial agents such as an antibiotic or other microbicidal or microbistatic agent. The contents of each compartment may be admixed prior to use or used sequentially in any order. Other antimicrobial agents include non-defensin peptides, a proteinase inhibitor, another defensin or a proteinaceous or chemical (non-proteinaceous) antimicrobial agent including an antibiotic.

Reference to a “plant defensin” means those defined herein with reference to Tables 1 and 2. As defined herein, a defensin includes a defensin having at least about 80% similarity to any one of SEQ ID NO:1 through 47. The 80% similarity is determined after optimal alignment and, where necessary, after appropriate spaces are used to optimize the alignment. By “at least 80%” or “at least about 80%” includes 80, 81, 82, 83, 84, 85, 86, 87, 88, 89, 90, 91, 92, 93, 94, 95, 96, 97, 98, 99 and 100%. In an embodiment, the defensin is defined by the consensus amino acid sequence SEQ ID NO:24 or a functional natural or synthetic derivative or variant thereof which includes a defensin having at least 80% similarity to SEQ ID NO: 25 after optimal alignment wherein SEQ ID NO:24 has an optional N-terminal alanine residue. Examples include SEQ ID NO;1, SEQ ID NO:2 and SEQ ID NO:3 and their N-terminal alanine variants, SEQ ID NO: 25 through 27, respectively). Some defensins use herein may be referred to as “naturally occurring”, “modified”, “variant” “mutated”, “synthetic derivative or variant” “chimeric” defensins. All such defensins retain antimicrobial activity.

Hence, taught herein is a method for inhibiting infection by a microorganism on or in in vivo tissue in a subject, the method comprising contacting the microorganism or tissue comprising the microorganism or administering to the subject an effective amount of plant defensin selected from SEQ ID NO:1 through 47 or a functional natural or synthetic derivative or variant thereof or a defensin having at least 80% similarity to any one of SEQ ID NO:1 through 47 after optimal alignment for a time and wider conditions sufficient to ameliorate symptoms of the infection. The defensin may have an N-terminal alanine residue such as SEQ ID NO:25 through 47.

The term “similarity” as used herein includes exact identity between compared sequences at the amino acid level. Where there is non-identity at the amino acid level, “similarity” includes amino acids that are nevertheless related to each other at the structural, functional, biochemical and/or conformational levels. In an embodiment, amino acid sequence comparisons are made at the level of identity rather than similarity.

Terms used to describe sequence relationships between two or more polypeptides include “reference sequence”, “comparison window”, “sequence similarity”, “sequence identity”, “percentage sequence similarity”, “percentage sequence identity”, “substantially similar” and “substantial identity”. A “reference sequence” is at least 12 but frequently 15 to 18 and often at least 25 or above, such as 30 amino acid residues in length. Because two polypeptides may each comprise (1) a sequence (i.e. only a portion of the complete amino acid sequence) that is similar between the two polypeptides, and (2) a sequence that is divergent between the two polypeptides, sequence comparisons between two (or more) polypeptides are typically performed by comparing sequences of the two polypeptides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA Clustal W2 and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (1997) Nucl. Acids. Res. 25(17):3389-3402. A detailed discussion of sequence analysis can be found in Unit 19.3 of Ausubel et al. (1994-1998) In: Current Protocols in Molecular Biology, John Wiley & Sons Inc. Other alignment software includes BWA (Li and Durbin (2010) Bioinformatics 26: 589-595) and Bowtie (Langmead et al (2009) Genome Biol 10:R25 and BLAT (Kent (2002) Genome Res 12:656-664).

The terms “sequence similarity” and “sequence identity” as used herein refer to the extent that sequences are identical or functionally or structurally similar on an amino acid-by-amino acid basis over a window of comparison. Thus, a “percentage of sequence identity”, for example, is calculated by comparing two optimally aligned sequences over the window of comparison, determining the number of positions at which the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by any suitable method or computer algorithm using standard defaults as used in the reference manual accompanying the software. Similar comments apply in relation to sequence similarity.

Some defensins used herein may be referred to herein as a “naturally occurring” defensin, a “modified” defensin, a “variant” defensin, a “mutated” defensin or a “chimeric” defensin, depending on its source.

In an embodiment, the defensin is a Class II Solanaceous defensin. In an embodiment, the defensin is modified at the loop region between the first β-strand (β-strand 1) and the α-helix at the N-terminal end portion of the defensin. In an embodiment, the loop region comprises the 6 amino acids N-terminal of the second invariant cysteine residue or its equivalent. This region is defined as “Loop1B”. A Class II Solanaceous defensin is distinguished from other defensins by a relatively conserved C-terminal end portion of the mature domain.

Included herein is the use of an artificially created defensin comprising a modified Class II Solanaceous defensin backbone wherein the loop region between β-strand 1 and the α-helix on the N-terminal end portion is modified by a single or multiple amino acid substitution, addition and/or deletion to generate a variant defensin which has anti-pathogen activity. In an embodiment, the loop region is Loop1B defined by the 6 amino acid residues N-terminal to the second invariant cysteine residue. Its equivalent region in any defensin is contemplated herein. In an embodiment, the artificially created defensin comprises a modified Class II defensin.

Examples of suitable defensins are defined by amino acid sequence (Table 1) and further characterized in Table 1. These comprise synthetic defensin variants such as HXP4 (SEQ ID NO:21), HXP34 (SEQ ID NO:22) and HXP35 (SEQ ID NO:23) or the same defensins with an N-terminal alanine residue (SEQ NO:45, 46 and, 47, respectively).

Taught herein is a method for inhibiting infection by a microorganism, on or in in vivo tissue in or on a subject, the method comprising contacting the microorganism or tissue comprising the microorganism or administering to the subject with an effective amount of a plant defensin selected from the group consisting of SEQ ID NO:1 through 47 or functional derivative or variant thereof. Generally, the defensin is applied for a time and under conditions sufficient to ameliorate the symptoms of the infection.

The defensin may be provided at a concentration of between 0.1% and 100% w/v, at a frequency of once a day, twice a day, once every two days, once a week, once every two weeks or once a month, for a period of one week, two weeks, three weeks, one month, two months, three months or up to 12 months.

In an optional embodiment, the defensin or its derivative or variant is used in combination with another antimicrobial agent. It is proposed that the defensin and the antimicrobial agent act in synergy. Examples of other agents include a non-defensin antimicrobial peptide, a proteinase inhibitor another defensin or a proteinaceous or non-proteinaceous chemical microbicide including an antibiotic.

Reference to synergy means that the inhibitory effect of a given defensin or other agent alone is greater when both are used together compared to either used alone. Greco et al. (1995) Pharmacol Rev. 47:331-385 define a category of synergy on the basis that the use of two agents in combination has greater activity relative to the additive effects when each is assayed alone. Hence, the definition adopted herein includes all such situations provided that the combined effect of the two agents acting together is greater than the sum of the individual agents acting alone. Furthermore, a combination of agents is deemed synergistic, as the term is intended herein, if there exists a set of conditions, including but not limited to concentrations, where the combined effect of the agents acting together is greater than the sum of the individual components acting alone. Richer (1987) Pestic Sci 19:309-315 describes a mathematical approach to establish proof of synergy. This approach uses Limpet's formula for comparing an observed level of inhibition (Io) in the combined presence of two inhibitor agents, X and Y, with an expected additive effect (Ee) resulting from each X or Y acting separately at the same respective concentrations as used to measure their combined effect. Additive percent inhibition, Ee, is calculated as X+Y−XY/100 where X and Y are expressed as percent inhibition. Synergism exits where Io>Ee.

Synergy may be expressed as a synergy scale. In an embodiment, a value of up to 14 represents no significant synergy such as 0, 1, 2, 3, 4, 5, 6, 7, 8, 9, 10, 11, 12, 13 or 14; a value of from 15 up to 29 represents low synergy such as 15, 16, 17, 18, 19, 20, 21, 22, 23, 24, 25, 26, 27, 28 or 29; a value of from 30 to 60 represents medium synergy such as 30, 31, 32, 33, 34, 35, 36, 37, 38, 39, 40, 41, 42, 43, 44, 45, 46, 47, 48, 49, 50, 51, 52, 53, 54, 56, 57, 58, 59 or 60; a value greater than 60 represents a high degree of synergy. By “greater than 60” includes from 61 to 100 including 61, 70, 80, 90 and 100 and any value in between.

The present method is useful in the treatment or prophylaxis of a subject having an infection on or in an in vivo tissue. The defensins defined by SEQ ID NO:1 through 47 are defined in Tables 1 and 2. The present invention encompasses the treatment of any internal surface tissue or membrane which is not external skin, hair or nails. Reference to “external skin” does not exclude subcutaneous layers of external skin or a surface wound. The term “subject” includes a human of any age or an animal such as a farm animal (e.g. sheep, pig, horse, cow, donkey, camel, llama, alpaca) or poultry bird (e.g. chicken, duck, turkey, pheasant, peacock), companion animal (e.g. dog or cat), laboratory test animal (e.g. mouse, rat, rabbit, guinea pig or hamster) or captive wild animal (e.g. wild goat).

The microorganism may be a fungus or a bacterium. Reference to a “fungus” includes dermatophytes, yeasts and non-dermatophytic molds (non-dermatophytes). Dermatophytes include Trichophyton species including Trichophyton rubrum, Trichophyton interdigitale, Trichophyton violaceum, Trichophyton tonsurans, Trichophyton soudanense and Trichophyton mentagrophytes, Microsporum fulvum, Epidermophyton floccosum and Microsporum gypseum. Yeasts encompasses Candida species including Candida albicans, Candida glabrate, Candida parasitosis, Candida tropicalis and Candida krusei. Cryptococcus species including Cryptococcus neoformans and Cryptococcus gattii, and Malassezia species including Malassezia globosa, Malassezia furfur, Malassezia dermatis and Malassezia restricts and Penicillium marneffei. Non-dermatophytic molds include species of Aspergillus including Aspergillus fumigatus, Aspergillus flavus, Aspergillus terreus and Aspergillus niger, Rhizopus species including Rhizopus oryzae, Neoscytalidium, Scopulariopsis, Acremonium, Fusarium species including Fusarium solani, Fusarium and oxysporum, Scytalidium and. Oomycetes include Pythium insidiosum. Reference to a bacterium includes Staphylococcus spp, Streptococcus ssp, Salmonella spp, Proteus spp, E. coli spp, Bacillus spp, Mycobacterium spp, Mycoplasma spp, Bacteroides spp, Fusobacterium spp.

The component or extract is administered with a pharmaceutical carrier, which is non toxic to cells and the individual.

The carrier may take a wide variety of forms depending on the form of preparation desired for administration, e.g., oral or parenteral (including intravenous) systemic. In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

Pharmaceutical compositions of the present invention used for therapy suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

The components and/or extracts identified of the present invention may be administered orally, parenterally or systemically (including subcutaneous injections, intravenous, intramuscular, intraperitoneal intrasternal injection, intranasal or infusion techniques), by inhalation spray, or rectally, in dosage unit formulations containing conventional non-toxic pharmaceutically acceptable carriers, adjuvants and vehicles.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The defensins of the invention may also be administered in intravenous (both bolus and infusion), intraperitoneal, subcutaneous, topical with or without occlusion, or intramuscular form, all using forms well known to those of ordinary skill in the pharmaceutical arts. When administered by injection, the injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters or polyethylene glycols, which are solid at ordinary temperatures, but liquidity and/or dissolve in the rectal cavity to release the drug.

The effective dosage of the defensins employed in therapy may vary depending on the particular compound employed, the mode of administration, the condition being treated and the severity of the condition being treated. Thus, the dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

The defensin may be administered directly to the infected site or provided systemically or by other convenient means generally for a time and under conditions sufficient to ameliorate the symptoms of infection.

Another aspect provides a protocol or method for treating or preventing an animal including a mammalian such as a human subject having in vivo tissue infected with a microorganism, the protocol or method comprising administering to the subject or site of infection an antimicrobial effective amount of a composition comprising the plant defensin for a time and under conditions sufficient to treat the infection.

The present defensin may be manufactured based on its amino acid sequence using standard stepwise addition of one or more amino acid residues using, for example, a peptide or protein synthesizer. Alternatively, the defensin is made by recombinant means. A recombinant defensin may include an additional alanine residue at its N-terminus. Hence, defensins contemplated herein may contain the N-terminus alanine residue.

In addition, the defensin may be subject to chemical modification to render the defensin a chemical analog. Such defensin analog, may exhibit greater stability or longer half life at the point of contact with the tissue.

Analogs contemplated herein include but are not limited to modification to side chains, incorporating of unnatural amino acids and/or their derivatives during peptide, polypeptide or protein synthesis and the use of crosslinkers and other methods which impose conformational constraints on the defensin molecule. This term also does not exclude modifications of the defensin, for example, glycosylations, acetylations, phosphorylations and the like. Included within the definition are, for example, defensins containing one or more analogs of an amino acid (including, for example, unnatural amino acids) or defensins with substituted linkages. Such analogs may have enhanced stability and/or penetrability.

Examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH4.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A bifunctional crosslinkers such as the bifunctional imido esters having (CH2)n spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of Cα and N α-methylamino acids, introduction of double bonds between Cα and Cβ atoms of amino acids and the formation of cyclic peptides or analogs by introducing covalent bonds such as forming an amide bond between the N and C termini, between two side chains or between a side chain and the N or C terminus.

Mimetics are another useful group of defensin analog. The term is intended to refer to a substance which has some chemical similarity to the defensin and mimics its antifungal activity. A peptide mimetic may be a peptide-containing molecule that mimics elements of protein secondary structure (Johnson et al., Peptide Turn Mimetics in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate activity actions.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The present disclosure illustratively described herein suitably may be practiced in the absence of any element or elements, limitation or limitations which is not specifically disclosed herein.

When a Markush group or other grouping is used herein, all individual members of the group and all combinations and sub-combinations possible of the group are intended to be individually included in the disclosure.

When a range is recited herein, it is intended that all sub-ranges within the stated range, and all integer values within the stated range, are intended, as if each sub-range and integer value was recited.

### EXAMPLES

Aspects disclosed and enabled herein are now described in the following non-limiting Example.

### Methods

Purification of Defensins from Pichia Pastoris

A single pPINK-defensin P. pastoris PichiaPink (Trademark) strain 1 colony is used to inoculate 25 mL of BMG medium (described in the Invitrogen Pichia Expression Manual) in a 250 mL flask and that is incubated over for 2-3 days in a 30° C. shaking incubator (140 rpm). The culture is used to inoculate 200 mL of BMG in a 1 L baffled flask which is placed in a 30° C. shaking incubator (140 rpm) overnight. The cells are harvested by centrifugation (1,500×g, 10 min, 4° C.) and resuspended into 1 L of BMM medium in a 5 L baffled flask and incubated in a 28° C. shaking incubator for 3 days (2 days for NaD1). The cultures are induced at t=24 and 48 h. The expression medium is separated from cells by centrifugation (6000 rpm, 20 min, 4° C.). The medium is adjusted to pH 3.0 before it is applied to an SP Sepharose column (1 cm×1 cm, Amersham Biosciences) pre-equilibrated with 100 mM potassium phosphate buffer, pH 6.0. The column is then washed with 100 mL of 100 mM potassium phosphate buffer, pH 6.0 (wash×2 for HXL004). Bound protein is eluted in 10×10 mL of 100 mM potassium phosphate buffer containing 500 mM NaCl. A dot blot is performed to identify factions with the highest concentration of eluted protein and those fractions are concentrated down to 1 mL using a centrifugal column and washed 5× using sterile milli Q ultrapure water. The protein concentration of Pichia-expressed defensin is determined using the bicinchoninic acid (BCA) protein assay (Pierce Chemical Co.) with bovine serum albumin (BSA) as the protein standard.

The defensins contemplated for use herein are listed in Tables 1 and 2 and include defensins having at least 80% similarity to any one of the listed defensins after optimal alignment.

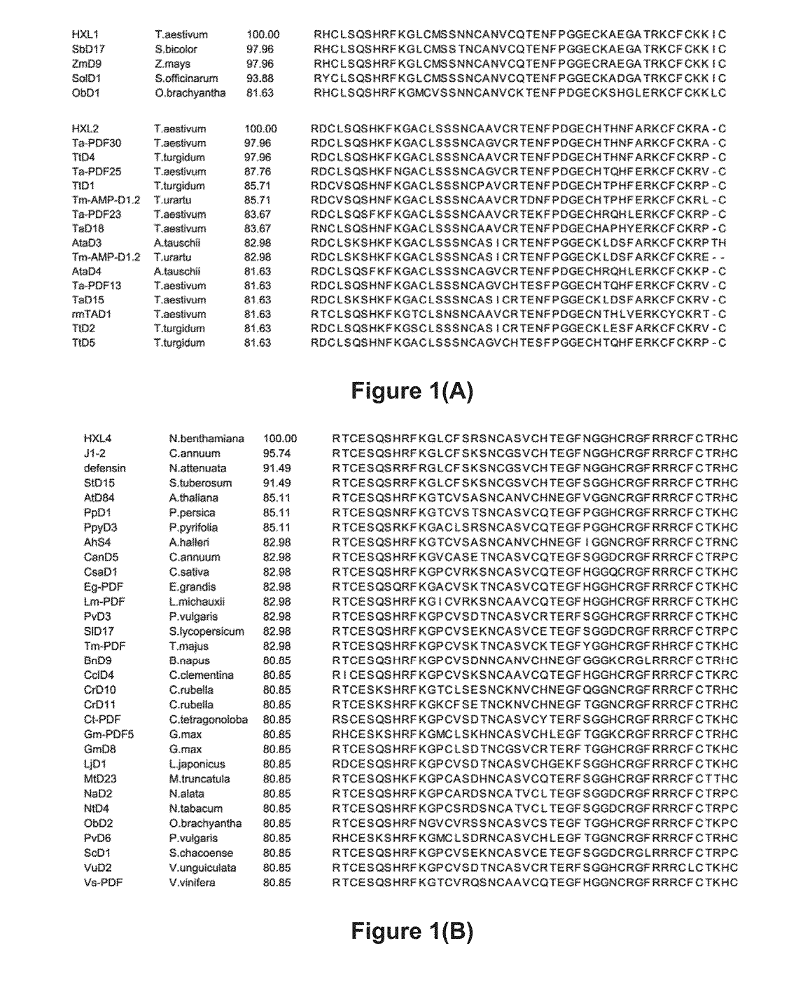

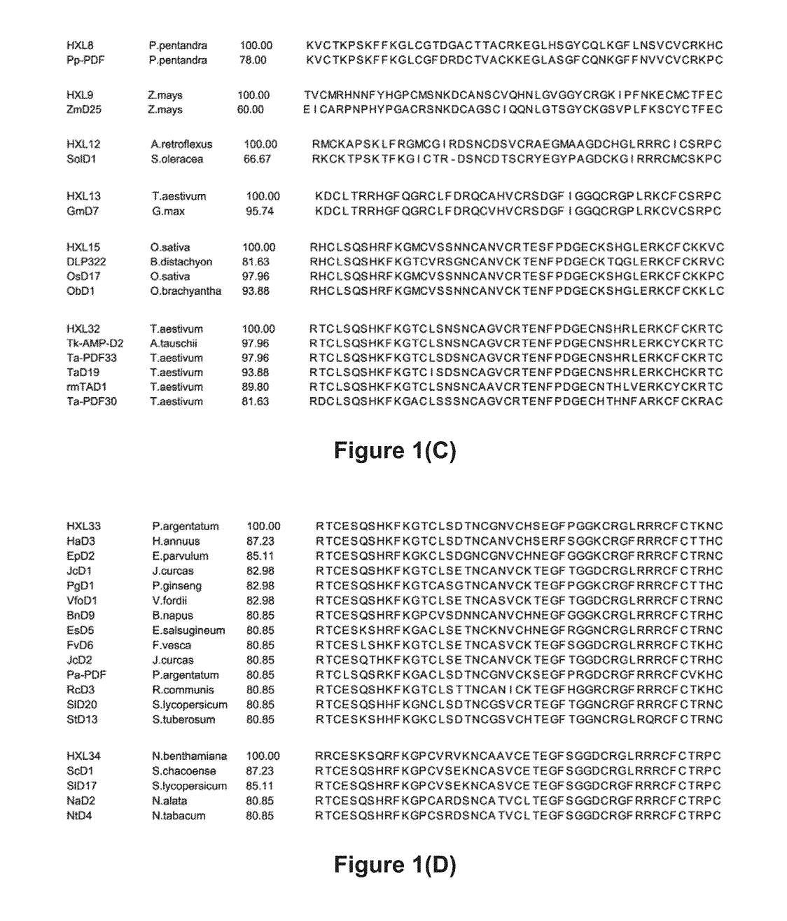

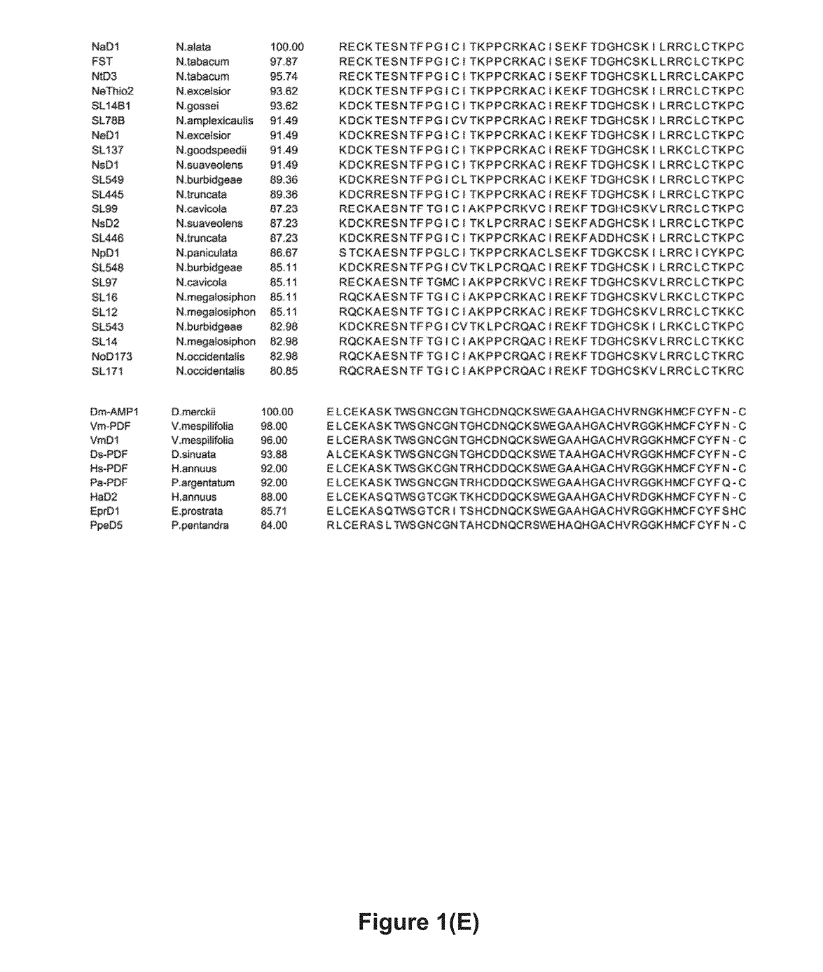

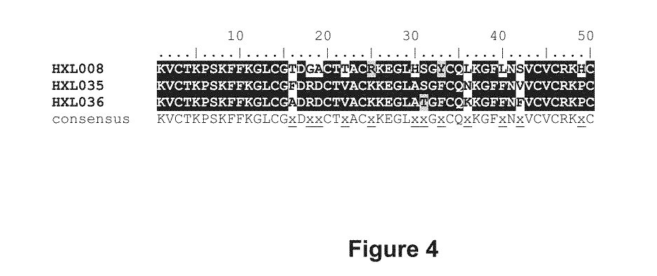

FIGS. 1(a) through (e) provide an alignment showing the similarity of amino acid sequences between a number of defensins. FIG. 4 provides an alignment of amino acids sequences for HXL008 (SEQ ID No: 1), HXL035 (SEQ ID No: 2), and HXL036 (SEQ ID No: 3). This alignment issued to generate a consensus amino acid sequence set for the in SEQ ID NO: 24. Any of these sequences may contain an optional N-terminal alanine residue.

### Example 1

### Inhibition of the Growth of Trichophyton Rubrum and Microsporum Fulvum in the Presence of a Plant Defensin

Plant defensins include a Solanaceous Class II defensin (NaD1), a modified Loop1B variant (HXP4) and Class I defensins (HXL001, HXL002, HXL004, HXL005, HXL008, HXL009, HXL012, HXL013, HXL015).

The inhibitory effects of the plant defensins on the growth of Trichophyton rubrum, T. interdigitale, Microsporum fulvum and C. albicans (all obtained from the National Mycology Reference Centre, South Australia Pathology at the Women's and Children's Hospital, Adelaide, Australia) are measured essentially as described by Broekaert et al. (1990) FEMS Microbiol Lett 69:55-59.

Spores of T. rubrum, T. interdigitale and M. fulvum are isolated from sporulating fungus growing on ½ strength Sabouraud dextrose agar. Spores were removed from the plates by the addition of ½ strength potato dextrose broth (PDB). C. albicans cells are grown in Yeast Peptone Broth (YPD) for 16 h. Spore and cell concentrations are measured using a hemocytometer.

Antifungal assays are conducted in 96 well microtitre plates essentially as herein described. Wells are loaded with 20 μL of filter sterilized (0.22 Ξ m syringe filter, Millipore) defensin (10× stock for each final concentration) or water and 80 μL of 5×10⁴ spores/mL (T. rubrum, T. interdigitale, M. fulvum) or 5,000 cells/mL (C. albicans) in ½ strength PDB. The plates are incubated at 30° C. Fungal growth is assayed by measuring optical density at 595 nm (A595) using a microtitre plate reader (SpectraMax Pro M2; Molecular Devices. Growth is allowed to proceed until the optical density (OD) of the fungus in the absence of any test defensin reached an OD of 0.2. Each test is performed in duplicate.

After incubation for 72 h, the media from wells containing clinical isolates of T. rubrum incubated with 100 g/mL HXL008 was plated onto fresh Sabouraud dextrose agar. Plates were incubated at 30° C. for 5 days to allow colonies to develop before being photographed.

The results of the inhibition assays are shown in Table 3. HXL005, HXL008 and HXL035 are the most effective plant defensins across the range of fungal pathogens. HXL001 and HXL009 did not display any activity at the concentrations tested. HXL002 and NaD2 are very poor inhibitors of M. fulvum and C. albicans. HXL004, HXL012, HXL013 and HXL015 display intermediate activity across the range of pathogens.

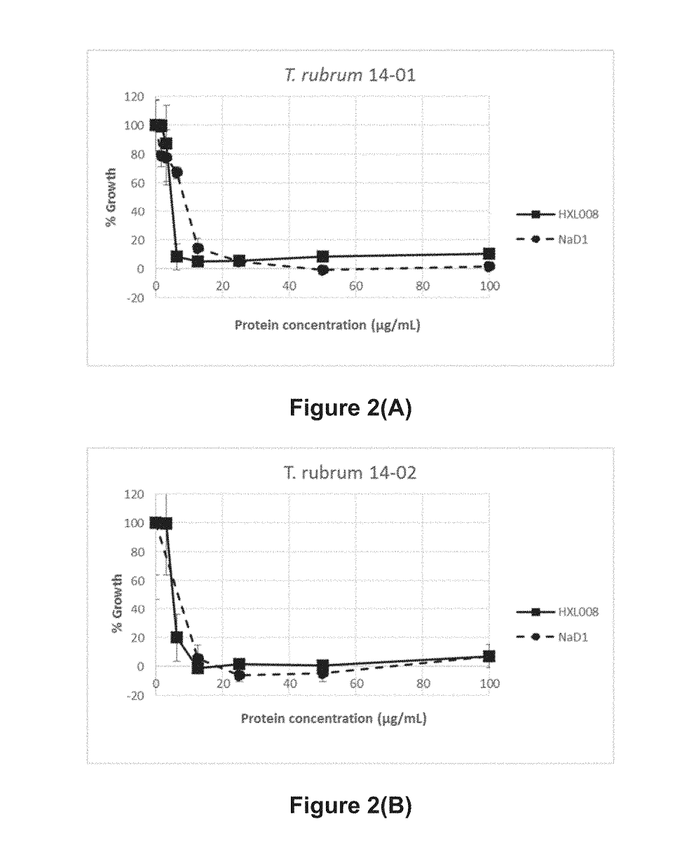

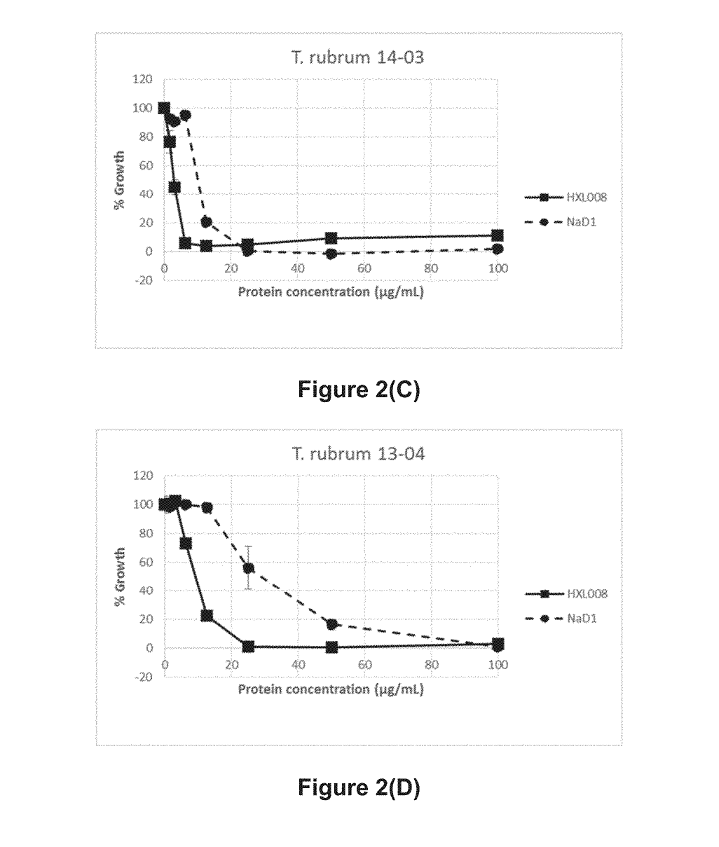

The results of inhibition of clinical isolates of T. rubrum by HXL008 (solid line) and NaD1 (dashed line) are shown in FIGS. 2(a) to 2(d). Both peptides inhibited fungal growth at low concentrations with IC50s of below 20 μg/mL against four clinical isolates. However, in all cases HXL008 inhibited growth at a lower concentration than NaD1.

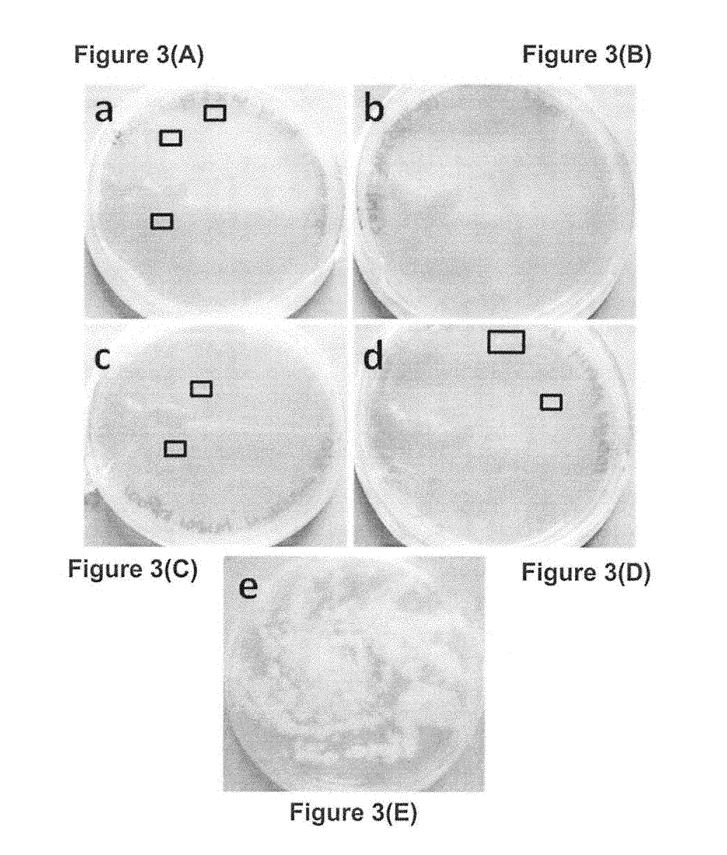

The results of cell survival assays for clinical isolates of T. rubrum are shown in FIGS. 3(a) to 3(e). Plates that had been inoculated with cells that had not been incubated with a plant defensin were almost completely covered in growth. In contrast, plates that were inoculated with cells had been incubated in the presence of HXL008 for 72 h only had 1-3 colonies indicating that almost all the cells were dead.

TABLE 3

| | | IC₅₀ | IC₅₀ | IC₅₀ |
|---|---|---|---|---|
| | IC₅₀ | against | against | against |
| | against T. rubrum | M. fulvum | T. interdigitale | T. albicans |
| Defensin | (μg/mL) | (μg/mL) | (μg/mL) | (μg/mL) |
| HXL001 | >100 | >100 | | 35 |
| HXL002 | | 50 | | 35 |
| HXL003 | | >100 | | >100 |
| HXL004 | 27.5 | 12 | 19 | 20 |
| HXL005 | 3 | 5 | 3.5 | 22 |
| HXL008 | 3 | 7 | 3.5 | 20 |
| HXL009 | | >100 | | >100 |
| HXL012 | 2 | 18 | 2 | 42 |
| HXL013 | 22 | 5.5 | | 20 |
| HXL015 | 10 | 12.5 | 3.5 | |
| HXL035 | 2 | 2 | 1 | 18 |
| NaD2 | | 38 | | 43 |
| NaD1 | 8 | 5.3 | 10 | 20 |

### Example 2

### Inhibition of the Growth of Fungal Pathogens in the Presence of a Plant Defensin

Plant defensins include a Solanaceous Class 11 defensin (NaD1) and Class 1 defensins (HXL001, HXL002, HXL003, HXL004, HXL005, HXL008, HXL009, HXL012, HXL013, HXL015, HXL035, NaD2).

The inhibitory effects of the plant defensins on the growth of Candida albicans, Aspergillus fumigatus (obtained from the National Mycology Reference Centre, South Australia Pathology at the Women's and Children's Hospital, Adelaide, Australia), C. glabrata, C. tropicalis, A. flavus, A. niger, A. fumigants, Cryptococcus neoformans and C. gattii (obtained from Dee Carter, University of Sydney, New South Wales, Australia) are measured essentially as described by Broekaert et al. (1990) FEMS Microbiol Lett 69:55-59.

Spores of A. flavus. A. niger and A. fumigatus are isolated from sporulating fungus growing on ½ strength Sabouraud dextrose agar. Spores were removed from the plates by the addition of ½ strength potato dextrose broth (PDB). C. albicans, C. glabrata, C. tropicalis, C. neoformans and C. gattii cells are grown in Yeast Peptone Broth (YPD) for 16 h. Spore and cell concentrations are measured using a hemocytometer.

Antifungal assays are conducted in 96 well microtitre plates essentially as herein described. Wells are loaded with 20 μL of filter sterilized (0.22 μm syringe filter, Millipore) defensin (10× stock for each final concentration) or water and 80 μL of 5×10⁴ spores/mL (A. flavus, A. niger, A. fumigatus), 5,000 cells/mL (C. albicans, C. glabrata, C. tropicalis) or 1×10⁶ cells/mL (C. neoformans, C. gattii) in ½ strength PDB. The plates are incubated at 30° C. Fungal growth is assayed by measuring optical density at 595 nm (A595) using a microtitre plate reader (SpectraMax Pro M2; Molecular Devices. Growth is allowed to proceed until the optical density (OD) of the fungus in the absence of any test defensin reached an OD of 0.2. Each test is performed in duplicate.

The results from the antifungal assay are presented in Table 4.

TABLE 4

| | IC₅₀ | IC₅₀ | IC₅₀ | IC₅₀ | IC₅₀ | IC₅₀ | IC₅₀ | IC₅₀ |
|---|---|---|---|---|---|---|---|---|
| | against | against | against | against | against | against | against | against |
| | C. albicans | C. glabrata | C. tropicalis | A. flavus | A. niger | A. fumigatus | C. neoformans | C. gattii |
| Defensin | (μg/mL) | (μg/mL) | (μg/mL) | (μg/mL) | (μg/mL) | (μg/mL) | (μg/mL) | (μg/mL) |
| HXL001 | 35 | 32 | 6 | >100 | >100 | >100 | 10 | 40 |
| HXL002 | 35 | 15 | 2 | 100 | 20 | 25 | 2.5 | 18 |
| HXL003 | >100 | >100 | 8 | >100 | >100 | >100 | 3 | 20 |
| HXL004 | 20 | 20 | 5 | >100 | 45 | 20 | 7 | 20 |
| HXL005 | 22 | 11 | 12 | 25 | 7.5 | 11 | 5 | 14 |
| HXL008 | 20 | 20 | 7 | 40 | 42 | 7.5 | 2.5 | 12 |
| HXL009 | >100 | >100 | 12 | >100 | >100 | >100 | 8 | 38 |
| HXL012 | 42 | >100 | 10 | 45 | 12.5 | 15 | 8 | 38 |
| HXL013 | 20 | 25 | 12 | >100 | >100 | >100 | 9 | 22 |
| HXL015 | 5 | | | | | | | |
| HXL035 | 18 | 10 | 6 | 25 | 6 | 7.5 | 5 | 20 |
| NaD2 | 43 | 23 | 6 | >100 | 50 | >100 | 4 | 20 |
| NaD1 | 20 | 14 | 2.5 | 20 | 16 | 8 | 3 | 5 |

### Example 3

### Inhibition of the Growth of Escherichia coli and Bacillus subtilis in the Presence of Plant Defensins

A single E. coli or B. subtilis colony was used to inoculate 5 ml of Luria-Bertani media and grown overnight at 37° C. The following day, the optical density of the culture was measured and the bacteria diluted to an optical density at 600 nm (OD₆₀₀) of 0.01 in Mueller-Hinton Broth. Diluted E. coli or B. subtilis were added to 96-well plates with defensins at concentrations of 20 μM, 10 μM, 5 μM, 2.5 μM, 1.25 μM, 0.625 μM or 0.3125 μM. Plates were read at OD₅₉₅ to obtain time zero data points. Plates were incubated at 37° C. for 18 hours before reading again at OD₅₉₅ to assess the amount of bacterial growth.

The results of inhibition of E. coli and B. subtilis are shown in Table 5. The plant defensin HXL004 inhibited the growth of both E. coli and B. subtilis with IC_{50S} of 2.5 and 2.6 μM respectively. This activity is similar to the LL37 control for E. coli. HXL012 and HXL013 inhibited growth of B. subtilis with IC_{50S} of 20 and 10 μM respectively. The defensins HXL001, HXL002, HXL003, HXL005, HXL008 and NaD1 did not inhibit the growth of E. coli or B. subtilis at the concentrations tested.

TABLE 5

| | IC₅₀ against | IC₅₀ against B. subtilis |
|---|---|---|
| | E. coli (μM) | (μM) |
| HXL001 | >20 | >20 |
| HXL002 | >20 | >20 |
| HXL003 | >20 | >20 |
| HXL004 | 2.5 | 2.6 |
| HXL005 | >20 | >20 |
| HXL008 | >20 | |
| HXL009 | >20 | >20 |
| HXL012 | >20 | 20 |
| HXL013 | >20 | 10 |
| NaD1 | >20 | |
| LL37 | 2.5 | |

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

### BIBLIOGRAPHY

Altschul et al. (1997) Nucl. Acids. Res. 25(17):3389-3402
Ausubel et al. (1994-1998) In: Current Protocols in Molecular Biology, John Wiley & Sons Inc
Bloch and Richardson (1991) FEBS Lett 279(1):101-104
Broekaert et al. (1990) FEMS Microbiol Lett 69:55-59
Cabral et al., (2003) Protein Express Purif 31(1):115-122
Colilla et al. (990) FEBS Lett 270(1-2):191-194
Fisher et al, (1981) The American Journal of Medicaine 71(4):571-7
Gomes et al. (2011) Clinical Microbiology Reviews 24(2):411-45
Greco et al, (1995) Pharmacol Rev 47:331-385
Janssen et al. (2003) Biochemistry 42(27):8214-8222
Johnson et al., Peptide Turn Mimetics in Biotechnology and Pharmacy, Pezzuto et al., Eds., Chapman and Hall, New York, 1993
Kent (2002) Genome Res 12: 656-664
Langmead et al (2009) Genome Biol 10:R25
Li and Durbin (2010) Bionformatics 26:589-595
Mitchell & Perfect (1995) Clinical Microbiology Reviews 8(4): 515-48)
Patterson et al. (2000) Medicine 79(4):250-60
Richer (1987) Pestic Sci 19:309-315
Sagaram et al., (2001) PLoS One 6.4: e18550
Singh et al. (2014) Journal of Oral and Maxillofacial Pathology 18(Suppl):S81-S85
Supparatpinyo et al. (1996) Lancet 344(8915):110-3
Thianprasit et al. (1996) Current Topics in Medical Mycology 7(1):43-54
Thomas (2003) Eye (London, England) 17(8):853-62
U.S. Pat. No. 6,911,577 (Pioneer Hi-Bred International, Inc. and E.I. DuPont DeNemours and Company)
Wanachiwanawin et al. (2004) Vaccine 22(27-28):3613-21
van der Weerden et al. (2013) Cell Mol Life Sci 70(19) 3545-3570
van der Weerden et al. (2013) Fungal Biol Rev 26:121-131
Vibhagool et al. (2003) Clinical Infectious Diseases 36(10):1329-31

1. A method for inhibiting infection of a microorganism on or in in vivo tissue in or on a subject, said method comprising contacting the microorganism or tissue comprising the microorganism or administering to the subject an effective amount of plant defensin selected from SEQ ID NO:1 through 47 or a functional natural or synthetic derivative or variant thereof or a defensin having at least 80% similarity to any one of SEQ ID NO:1 through 47 after optimal alignment for a time and under conditions sufficient to ameliorate symptoms of the infection.

2. The method of claim 1 wherein the microorganism is a fungus.

3. The method of claim 1 wherein the microorganism is a bacterium.

4. The method of claim 1 wherein the infection leads to a condition selected from aspergillosis, Candida infection of a mucosal membrane, systemic candidiasis, cryptococcosis, subcutaneous skin layer infection, gastroenteritis, respiratory infection and an infection leading to a sexually transmitted disease.

5. The method of claim 1 wherein the defensin is coated onto a medical device or condom.

6. The method of claim 1 wherein the defensin is defined by the consensus amino acid sequence SEQ ID NO:24.

7. The method of claim 6 wherein the defensin is selected from the group consisting of SEQ ID NO:1, SEQ ID NO:2 and SEQ ID NO:3 HX NO: or a functional natural or synthetic derivative or variant thereof which includes a defensin having at least 80% similarity to any one of SEQ ID NO:1, SEQ ID NO:2 and SEQ ID NO:3 after optimal alignment, each with an optional N-terminal alanine.

8. The method of claim 7 wherein a defensin with an N-terminal alanine is selected from the group consisting of SEQ ID NO:25, SEQ ID NO:26, SEQ ID NO:27.

9. The method of claim 1 wherein the defensin variant comprises a Loop1B from a Class I defensin replacing the corresponding Loop1B from a Solanaceous Class II defensin.

10. The method of claim 9 wherein the defensin variant is selected from the list consisting of HXP4, HXP34 and HXP35.

11. The method of claim 1 wherein the defensin is used in synergistic combination with an antimicrobial agent.

12. The method of claim 11 wherein the antimicrobial agent is a peptide of from about 0.4 to about 12 kD or a proteinase inhibitor.

13. The method of claim 1 wherein the subject is a human.

14. A topical coating for a medical device or condom comprising a plant defensin selected from SEQ ID NO:1 through 47 or a functional natural or synthetic derivative or variant thereof or a defensin having at least 80% similarity to any one of SEQ ID NO:1 through 50 after optimal alignment.

15. Use of a plant defensin selected from SEQ ID NO:1 through 47 or a functional natural or synthetic derivative or valiant thereof or a defensin having at least 80% similarity to any of SEQ ID NO:1 through 47 after optimal alignment in the manufacture of a medicament for the treatment or prophylaxis of microbial infection on or in an in vivo tissue in a human or animal subject.
